(19) United States
(12) Patent Application Publication
(10) Pub. No.: US 2023/0069802 A1
(43) Pub. Date: Mar. 2, 2023

(54) Recording Medium and Image Playback Method

(71) Applicant: Anaut Inc., Yokohama-shi, Kanagawa (JP)

(72) Inventors: Naoki Kitamura, Tokyo (JP); Yuta Kumazu, Yokohama-shi, Kanagawa (JP); Nao Kobayashi, Tokyo (JP)

(21) Appl. No.: 17/794,441

(22) PCT Filed: Nov. 5, 2020
(86) PCT No.: PCT/JP2020/041295

§ 371 Date: Jul. 21, 2022

(30) Foreign Application Priority Data
Jan. 30, 2020 (JP) PCT/JP2020/003482

(51) Int. Cl. A61B 1/00; G06T 7/00; A61B 5/02

(52) U.S. Cl. CPC A61B 1/0005; G06T 7/0016; A61B 5/02042; G06T 2207/10068

(57) ABSTRACT

A non-transitory recording medium recoding a program that causes a computer to execute processing includes: recording operation field images obtained by chronologically shooting an operation field under endoscopic surgery; determining presence or absence of a predetermined or larger amount of bleeding based on the operation field images; and playing back partial images according to a set playback mode in a time range from a time before a start of bleeding to the time after the bleeding among the recorded operation field images, when it is determined that the predetermined or larger amount of bleeding is present.

### CROSS-REFERENCE TO RELATED APPLICATIONS

This application is the national phase of PCT International Application No. PCT/JP2020/041295 which has an International filing date of Nov. 5, 2020 and designated the United States of America.

### FIELD

The present invention relates to a computer program and an image playback method.

### BACKGROUND

Patent Document 1 (Japanese Patent Application Laid-Open No. 2011-36370) proposes playback of a part of the moving image of a surgical operation.

In Patent Document 1, though checking for a necessary part through playback based on feature points is made possible, mere playback of a scene in which an incident such as bleeding occurs often makes it difficult to grasp a location of the cause of the incident.

### SUMMARY

The present invention is made in view of such a background and aims at provision of a technique that allows for easy understanding of an event during a surgical operation.

A computer program according to one aspect of the present invention causes a computer to execute processing of recording operation field images obtained by chronologically shooting an operation field under endoscopic surgery, determining presence or absence of a predetermined or larger amount of bleeding based on the operation field images, and playing back partial images according to a set playback mode in a time range from a time before a start of bleeding to the time after the bleeding among the recorded operation field images, when it is determined that the predetermined or larger amount of bleeding is present.

According to the present invention, an event during a surgical operation is made easily understandable.

The above and further objects and features of the invention will more fully be apparent from the following detailed description with accompanying drawings.

### BRIEF DESCRIPTION OF DRAWINGS

FIG. 1 is a conceptual view illustrating the outline of a surgical operation system according to a present embodiment;

FIG. 2 is a block diagram illustrating the outline of the surgical operation system according to the present embodiment;

FIG. 3 is a block diagram illustrating an example of the hardware configuration of a control unit 30;

FIG. 4 is a block diagram illustrating an example of the software configuration of the control unit 30;

FIG. 5 is a block diagram illustrating the outline of the configuration of an image M acquired from an endoscope camera 13;

FIG. 6 illustrates occurrence of an event;

FIG. 7 illustrates a frame F1 at the time of occurrence of an event;

FIG. 8 is an explanatory view illustrating an example of the frame F1 at the event occurrence time;

FIG. 9 is an explanatory view illustrating an example of a display 21 with a wipe image W displayed;

FIG. 10 is an explanatory view illustrating processing executed in a surgical operation system in the present embodiment;

FIG. 11 is an explanatory view illustrating an example of the software configuration of a control unit 30 according to Embodiment 2;

FIG. 12 is an explanatory view illustrating a flowchart of the processing according to Embodiment 2;

FIG. 13 is an explanatory view illustrating an example of the software configuration of a control unit 30 according to Embodiment 3;

FIG. 14 is a flowchart of the processing executed by the control unit 30 according to Embodiment 3;

FIG. 15 is an explanatory view illustrating an example of the software configuration of a control unit 30 according to Embodiment 4;

FIG. 16 is flowchart of the processing executed by the control unit 30 according to Embodiment 4;

FIG. 17 is an explanatory view illustrating an overview of a surgical operation system 1 according to Embodiment 5;

FIG. 18 is a flowchart of the processing executed by a control unit 30 according to Embodiment 6;

FIG. 19 is an explanatory view illustrating one example of a playback mode;

FIG. 20 is a flowchart of the processing executed by a control unit 30 according to Embodiment 7;

FIG. 21 is an explanatory view illustrating a display example of a thumbnail showing a bleeding scene;

FIG. 22 is a flowchart of the processing executed by a control unit according to Embodiment 8;

FIG. 23 is a display example of a graph showing a chronological variation of a bleeding amount; and

FIG. 24 is a flowchart of the processing executed by a control unit 30 according to Embodiment 10.

### DESCRIPTION OF EMBODIMENTS

The present invention is described with the contents of embodiments listed up. The present invention has a configuration as below, for example.

### Clause 1

A moving image playback mode comprising:

a moving image acquisition unit that acquires a moving image obtained by capturing an image of an operation field of an endoscopic surgery;

an event detection unit that detects a predetermined event in the operation field by analyzing the moving image;

a partial moving image extraction unit that extracts a partial moving image shot during a period including a detection point of the event from the moving image; and an event playback unit that plays back the partial moving image according to each of a plurality of playback modes.

### Clause 2

The moving image playback mode according to Clause 1, wherein

the playback modes include at least two of a normal playback, a slow playback, a repetitive playback and a reverse playback.

### Clause 3

The moving image playback mode according to Clause 1, wherein

the event playback unit selects a type of the plurality of playback modes according to at least any one of a type of an organ as a subject to be operated, an operative method related to a procedure, and a parameter related to the event.

### Clause 4

The moving image playback mode according to Clause 1, wherein

the event playback unit selects at least one of a playback speed related to the playback modes, a time duration from the detection time t0 a start time of the period, a time duration from the detection time t0 an end point of the period and a playback direction according to at least one of a type of an organ as an subject to be operated, an operative method related to a procedure and a parameter related to the event.

### Clause 5

The moving image playback mode according to Clause 1, wherein

the event is bleeding from the subject to be operated,

the event detection unit detects a bleeding amount,

the playback modes include at least the slow playback, and

the event playback unit determines a playback speed of the slow playback depending on at least the bleeding amount.

### Clause 6

The surgical operation support system according to Clause 1, wherein

the period corresponds to a first period, and

the event playback unit plays back the partial moving image for a second period within a first period which is the period described above at a playback speed lower than that in other periods, the second period being shorter than the first period and including the detection time.

### Clause 7

The surgical operation support system according to Clause 1, further comprising a setting unit that sets at least one of a type of the plurality of playback modes, a playback speed related to the playback modes, a time duration from the detection time t0 a start time of the period, a time duration from the detection time t0 an end time of the period and a playback direction.

Clause 8

The surgical operation support system according to Clause 7, wherein

the setting unit changes at least one of a type of the plurality of playback modes, a playback speed related to the playback modes, a time duration from the detection time t0 a start time of the period, a time duration from the detection time t0 an end time of the period and a playback direction after start of playback by the event playback unit.

### <Outline of System>

Hereinafter, a surgical operation system according to one embodiment of the present invention will be described. A surgical operation support system according to the present embodiment is for supporting an endoscopic surgery and can also be a moving image playback mode for outputting a moving image obtained by specifically capturing an image of an operation field and playing back, in the case where an event such as local bleeding, for example, occurs, a scene including the time when the event occurs.

### Embodiment 1

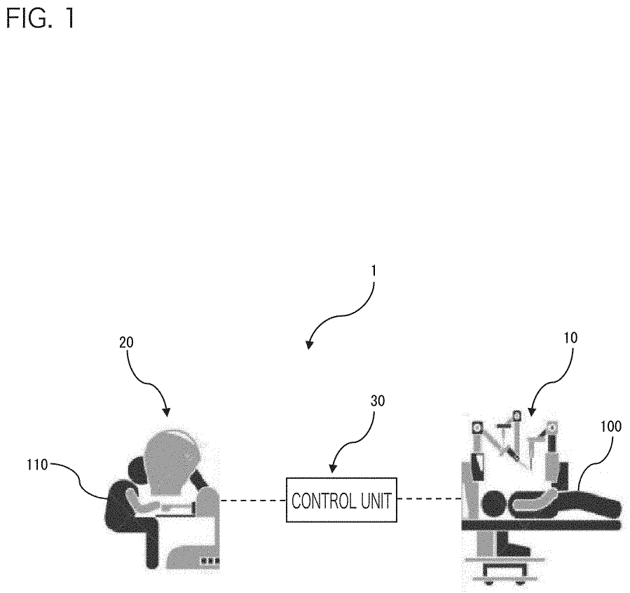

FIG. 1 is a conceptual view illustrating the outline of a surgical operation system according to the present embodiment. As illustrated in the drawing, a surgical operation system 1 includes an operation unit 10, an operation unit 20 and a control unit 30 for controlling the operation unit 10 and the operation unit 20.

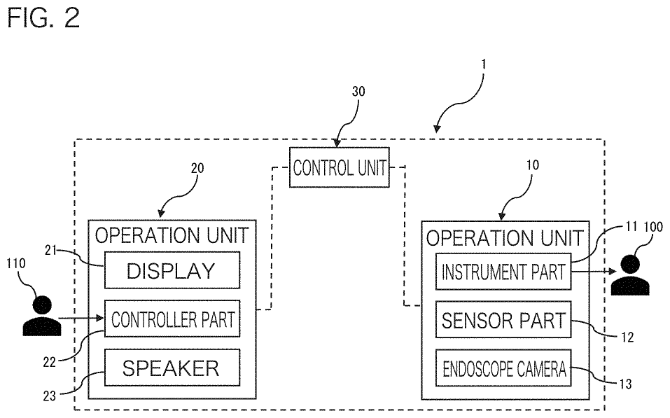

FIG. 2 is a block diagram illustrating the outline of a surgical operation system according to the present embodiment.

The operation unit 10 is for performing a surgical procedure on a patient 100 as a subject to be operated and includes an instrument part 11 as a procedure unit, a sensor part 12 as a procedure unit likewise and an endoscope camera 13 as an image acquisition unit in the present embodiment.

The instrument part 11 is constructed by a movable arm and a surgical instrument attached to the distal end of the movable arm. Though as surgical instruments, for example, a scalpel (electric scalpel or the like), scissors, forceps, a needle holder, tweezers and like are assumed, various surgical instruments other than the ones described above are attachable according to the applications.

The sensor part 12 employs various sensors such as a pressure sensor, a gyro sensor, an acceleration sensor, a temperature sensor or the like for detecting the state of the instrument part 11, for example.

The configuration of the endoscope camera 13 will be described later.

The operation unit 20 is for accepting an operation performed on the operation unit 10 by an operating surgeon 110 as an operator and includes a display 21 as a display part, a controller part 22 and a speaker 23 in the present embodiment. Furthermore, the operation unit 20 may be provided with a microphone for accepting voice instructions from the operating surgeon 110.

The display 21 is for providing the operating surgeon 110 with various information, and employs a technique of implementing a Head Mount Display (HMD) of VR (Virtual Reality) in the present embodiment, which enables stereoscopic views of an area to be viewed by utilizing parallax of both of the eyes of the operating surgeon 110.

The controller part 22 is implemented by an input device such as a joystick, a foot pedal and the like in the present embodiment, and the speaker 23 provides the operating surgeon 110 with various information by voice or the like.

### <Hardware of Control Unit 30>

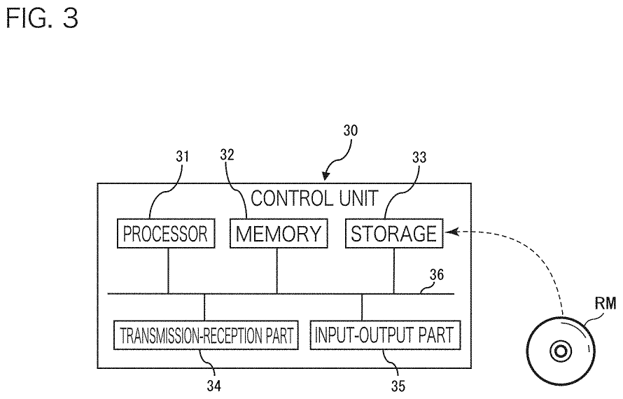

FIG. 3 is a block diagram illustrating an example of a hardware configuration of the control unit 30. As illustrated in the drawing, the control unit 30 includes as main components a processor 31, a memory 32, a storage 33, a transmission-reception part 34 and an input-output part 35, which are electrically connected to each other through buses 36.

The processor 31 is an arithmetic device for controlling the operation of the control unit 30 and performing control of data transmission and reception between the components as well as processing or the like necessary for execution of an application program.

The processor 31 is, for example, a CPU (Central Processing Unit) in the present embodiment and performs each processing by executing the application program or the like stored in the storage 33 to be described later and developing it on the memory 32.

The memory 32 is provided with a main storage formed of a volatile storage such as a DRAM (Dynamic Random Access Memory) and an auxiliary storage formed of a nonvolatile storage device such as a flash memory and an HDD (Hard Disc Drive).

This memory 32 is used as a work area of the processor 31 while it stores Basic Input/Output System (BIOS) to be executed upon startup of the control unit 30 as well as various setting information.

The storage 33 stores the application program, data used for various processing and the like. In the present embodiment, an image processing program for performing various processing is stored. The image processing program is provided by a non-transitory recording medium RM in which a program is readably recorded, for example. The processor 31 may read a desired program from the recording medium RM using a reading device (not illustrated) and may merely store the read program in the storage 33. The details of the image processing program will be described later.

The transmission-reception part 34 connects the control unit 30 to the Internet. The transmission-reception part 34 may be equipped with a near field communication interface such as Bluetooth (registered trademark) and Bluetooth Low Energy (BLE).

In the present embodiment, the control unit 30 is connected to the operation unit 10 and the operation unit 20 through the transmission-reception part 34.

The input-output part 35 is connected, as necessary, to information input devices such as a keyboard and a mouse and an output device such as a display.

The bus 36 transfers an address signal, a data signal and various control signals, for example, among the processor 31, the memory 32, the storage 33, the transmission-reception part 34 and the input-output part 35 that are connected to each other.

### <Software of Control Unit 30>

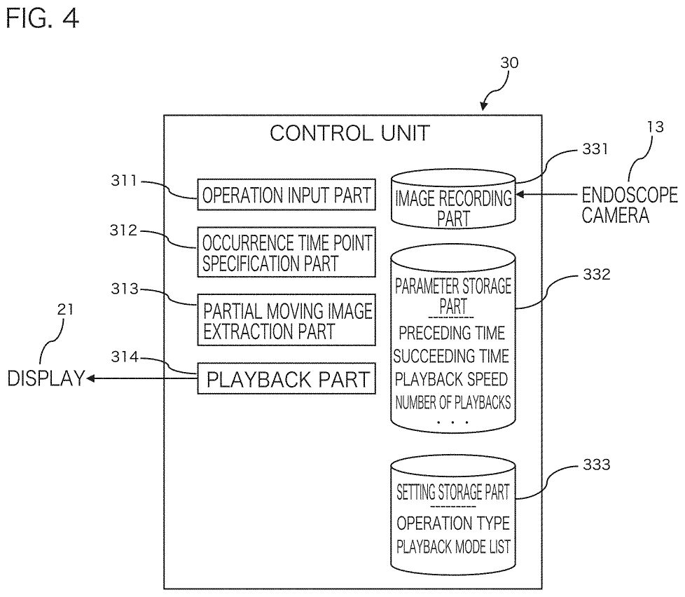

FIG. 4 is a block diagram illustrating an example of the software configuration of the control unit 30. As illustrated in the drawing, the control unit 30 includes an operation input part 311, an occurrence time specification part 312, a partial moving image extraction part 313, a playback part 314, a image recording part 331, a parameter storage part 332 and a setting storage part 333.

The image recording part 331 records as a recorded image M1 an image M that is acquired by the endoscope camera 13 and displayed in real time on the display 21.

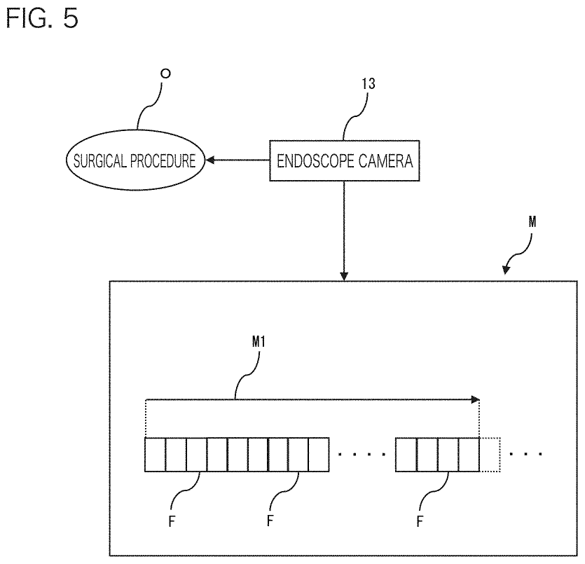

FIG. 5 is a block diagram illustrating the outline of the configuration of the image M acquired from the endoscope camera 13. As illustrated in the drawing, the endoscope camera 13 is for capturing an image of a surgical procedure O performed on the patient 100 with the instrument part 11 and acquires the shot images as the image M. The image M acquired by the endoscope camera 13 consists of multiple frames F and is successively displayed on the display 21 in real time acquired while the image M displayed on the display 21 is recorded in the image recording part 331 as the recorded image Ml.

The surgical operation system according to the present embodiment with such a configuration is to endoscopically perform minimally invasive endoscopic surgery by making more than one small holes of approximately 3 to 10 mm in the body of the patient 100, which reach the abdominal cavity or thoracic cavity of the patient 100, and by inserting the endoscope camera 13 and a surgical instrument of the instrument part 11 through the holes without incision of the body.

The operation input part 311 accepts various operations that are input through the operation unit 20 by the operating surgeon 110.

The occurrence time specification part 312 specifies a time when an event occurs (hereinafter referred to as an event occurrence time) from the recorded image Ml. The occurrence time specification part 312 performs an image analysis on each of the frames F that constitute the recorded image M1 and extracts the feature quantity (red color of bleeding, for example) of an image representing an event to thereby determine whether or not an event occurs in the frame of interest and specify the time corresponding to the foremost frame out of the frames F shot during a series of the same event as an event occurrence time.

The occurrence time specification part 312 can be implemented by an artificial intelligence module for autonomously specifying event-related frames by machine learning, for example. As methods of performing machine learning, various algorithms such as a neural network, a random forest, a support vector machine (SVM) and the like can appropriately be used.

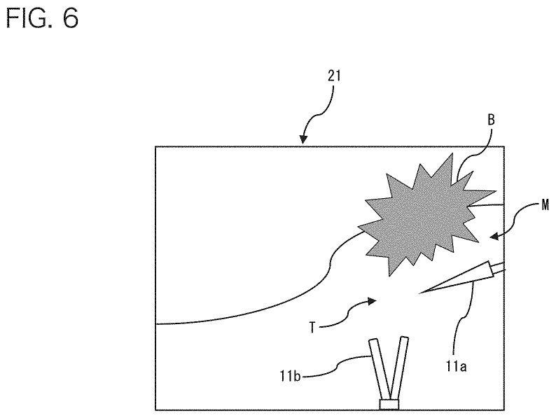

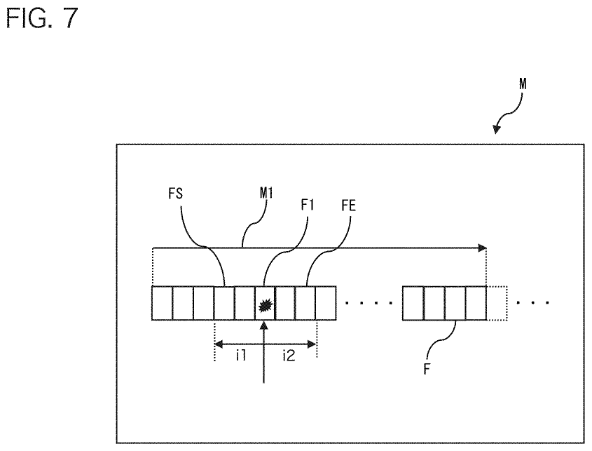

FIG. 6 illustrates occurrence of an event. FIG. 7 illustrates a frame F1 at the time of occurrence of an event.

As illustrated in FIG. 6, when an unexpected accident (event) such as local bleeding, for example, occurs to the patient 100 during a surgical procedure performed on an affected area T of the patient 100 with the surgical instruments 11a, 11b of the instrument part 11 of the operation unit 10 and bleeding B appears on the image M displayed in real time on the display 21, the occurrence time specification part 312 can specify the frame F1 in which the occurrence of bleeding B is recorded out of the frames F that constitute the recorded image Ml, as illustrated in FIG. 7. In the frame F1 corresponding to the event occurrence, the initial stage of bleeding B is recorded where blood has just started to ooze before spreading, i. e. the state immediately after the bleeding B occurs.

The partial moving image extraction part 313 extracts a partial moving image (frames included in this period) shot during a period including the event occurrence time. In the example of FIG. 7, regarding the frame FS at a time a time i1 before the frame F1 at the event occurrence time t0 as a start frame and regarding the frame FE at a time a time i2 after the frame F1 at the occurrence time t0 as an end frame, the partial moving image extraction part 313 can extract the frames from the start frame to the end frame as a partial moving image. That is, the partial moving image extraction part 313 can extract from the recorded image M1 a partial moving image in a period from the start time t0−i1 to the end time t0+i2.

The parameter storage part 332 stores various parameters such as times i1, i2 and the like. In the example of FIG. 4, the parameter storage part 332 stores parameters such as a preceding time, a succeeding time, a playback speed, the number of playbacks and the like. For example, the preceding time is the time i1 while the succeeding time is the time i2. The playback speed is a playback speed when the playback part 314 to be described later performs a slow playback of a partial moving image. The number of playbacks is the number of repetitions when the playback part 314 to be described later performs a repetitive playback of a partial image.

The playback part 314 plays back a partial moving image. In the present embodiment, the playback part 314 plays back a partial moving image using multiple playback modes. The multiple playback modes include at least two of a normal playback, a slow playback, a repetitive playback and a reverse playback. That is, the playback part 314 can play back a scene including the event occurrence time multiple times while varying the playback modes. Various parameters related to the playback modes (playback speed for the slow playback, the number of playbacks for the repetitive playback, for example) are stored in the parameter storage part 332.

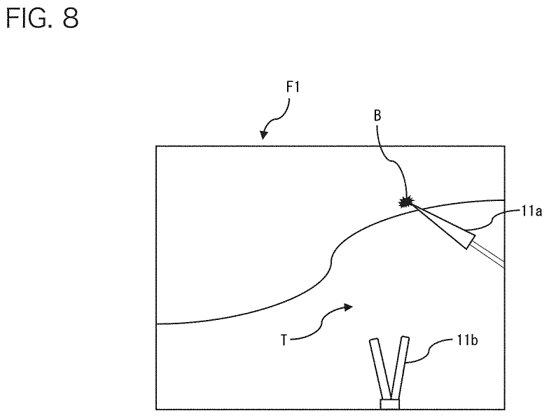

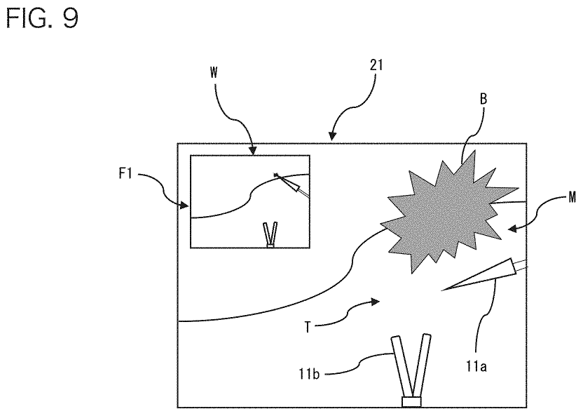

The playback part 314 can display a partial image as a wipe image on the display 21 that the operating surgeon 110 is viewing. FIG. 8 is an explanatory view illustrating an example of the frame F1 at the time of occurrence of an event. FIG. 8 illustrates an example at a time when occurrence of bleeding B is recognized. FIG. 9 is an explanatory view illustrating an example of the display 21 with a wipe image W displayed. In the example of FIG. 9, the frame F1 at the time of occurrence of an event is displayed as a wipe image W.

Moreover, the playback part 314 can play back a partial moving image by a combination of different playback modes depending on the types of the operation performed with the surgical instrument of the instrument part 11. A playback mode to be used is stored in the setting storage part 333 to be described later.

In the example of FIG. 4, the setting storage part 333 stores multiple playback modes (list) by associating them with the operation types. The operation types include resection, abrasion, traction, suture, suction and the like, and these can be specified by an image analysis using machine learning, for example. These operation types may be specified depending on the kind of the surgical instrument attached to the instrument part 11 or an operation input signal input by the operating surgeon 110 that is accepted through the operation input part 311.

### <Processing>

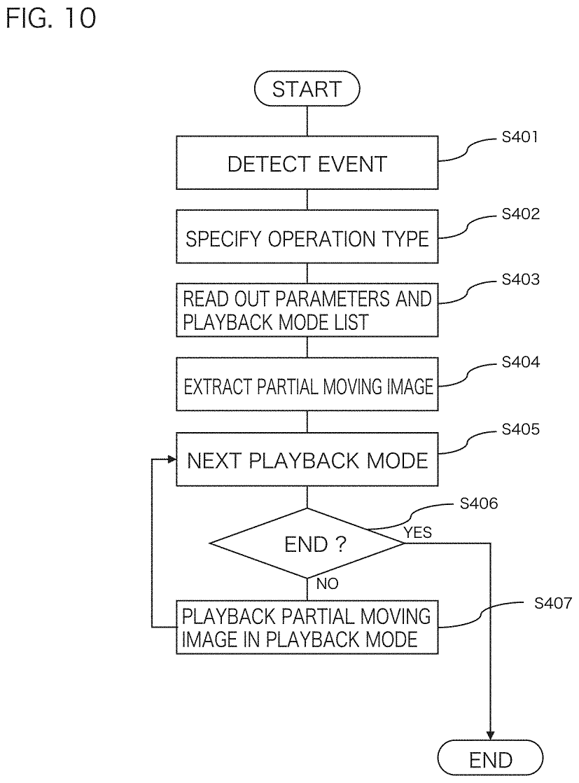

FIG. 10 is an explanatory view illustrating processing executed in the surgical operation system according to the present embodiment. In the drawings, the flow of the operation of the control unit 30 is shown.

The occurrence time specification part 312 specifies a time (frame) when an event occurs from the recorded image M1 (S401) and specifies an operation type of a surgical instrument of the instrument part 11 from the recorded image M1 (S402). For example, the occurrence time specification part 312 can specify the operation type by analyzing an image.

The partial moving image extraction part 313 respectively reads out parameters and a playback mode list from the parameter storage part 332 and the setting storage part 333 (S403), calculates a extraction period (t0−i1 to t0+i2) from the preceding time (i1) and the succeeding time (i2) of the read parameters and the specified event occurrence time t0 and extracts a partial moving image shot during the calculated period from the recorded image M1 (S404).

The playback part 314 extracts a next playback mode from the playback modes included in the playback mode list (S405), and plays back the partial moving image in the next playback mode (S407) when the next playback mode is present (S406: NO). Here, the parameters in the playback mode (playback speed of the slow playback, the number of repetitions of the repetitive playback and the like) employs the read parameters described above. The playback part 314 may output the partial image as a wipe image W as illustrated in FIG. 9 superimposed on the real time image acquired by the endoscope camera 13, or may output the partial image to the sub display having been provided separately from the display 21.

As in the description above, according to the surgical operation system of the present embodiment, in the case where an event (bleeding, for example) occurring in a space (abdominal cavity, for example) of a subject to be operated is detected, a partial moving image shot during a period including the event occurrence time, i.e., an event occurrence scene can be output. By checking the wipe image W as illustrated in FIG. 9 showing the moving image shot during the period including the initial time when the bleeding B started (event occurrence time) in a state where the bleeding B has occurred and spread as in the example FIG. 6, the operating surgeon 110 can easily specify a bleeding site where the bleeding B occurs, for example. The reason for occurrence of the bleeding B, i.e., the cause of the bleeding B can also be specified, which enables an appropriate procedure. The following reasons can be conceivable for bleeding, for example. That is, assuming that bleeding occurs after a tissue in the body is snipped with a resection tool and activated to break the tissue, the process from breakage of the tissue to the spread of bleeding from the bleeding point occurs in an extremely short time period though the operation until snipping and the time during activation can be grasped at a normal speed. Thus, reducing the playback speed for this bleeding scene facilitates understanding of the scene. Furthermore, a reverse playback allows the spread blood to converge to one point, which facilitates understanding of the location of the bleeding point. Thus, partial reduction of the playback speed and reverse playback make it easy to specify the event occurrence location and the event occurrence cause.

Moreover, according to the surgical operation system of the present embodiment, a partial moving image shot during a period including an event occurrence time such as bleeding B or the like can be checked by multiple playback modes. Accordingly, displaying the past scene multiple times as in the present embodiment enables easy and reliable grasping of the event occurrence position and the event occurrence cause in comparison with the case of checking the past scene only once. Additionally, by playing back the same scene in the different playback modes as in the present embodiment, the identical event can be checked in different way of viewing, so that the event occurrence location (bleeding site, for example) and the event occurrence factor (the cause of bleeding, for example) can be grasped more easily and reliably.

### Embodiment 2

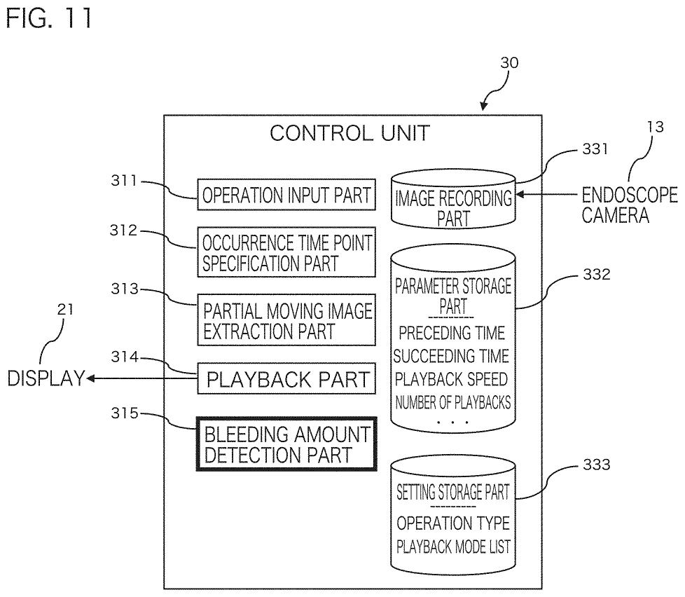

FIG. 11 is an explanatory view illustrating an example of the software configuration of a control unit 30 according to Embodiment 2. Though the playback speed of the slow playback (or fast-forward playback) is assumed to have been set to the parameter storage part 32 as the parameters of Embodiment 1 described above, a playback speed can be changed depending on the amount of bleeding in Embodiment 2.

The control unit 30 according to Embodiment 2 includes a bleeding amount detection part 315 in addition to the configuration of the control unit 30 in Embodiment 1 described above (see FIG. 4). The bleeding amount detection part 315 analyzes the image of each of the frames F that constitute the recorded image M1 and estimates the amount of bleeding included in the images. The bleeding amount detection part 315 can calculate the amount of bleeding or the like depending on the number of pixels of a color of blood included in the image (or a bleeding area, i.e., the ratio of the region covered with bleeding to the target region to be operated), for example. The playback part 314 according to Embodiment 2 changes the playback speed according to the amount of bleeding. More specifically, the playback part 314 can reduce the playback speed as the amount of bleeding (or the bleeding area) increases. In addition, the playback part 314 can reduce the playback speed as a chronological variation (an increasing amount per unit time of the bleeding amount, for example) of the bleeding amount (or the bleeding area) increases.

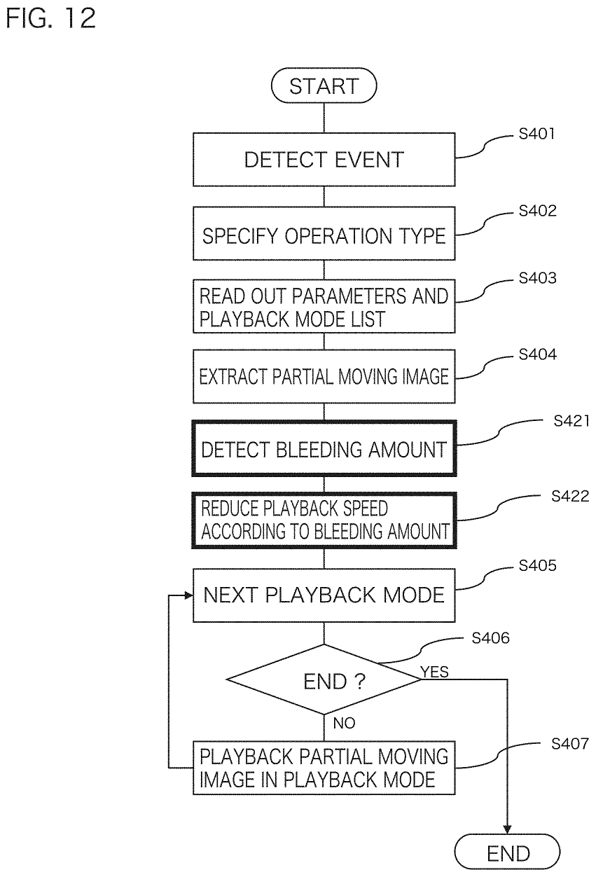

FIG. 12 is an explanatory view illustrating a flowchart of the processing according to Embodiment 2. The difference from the processing according to Embodiment 1 (FIG. 10) will be described. In Embodiment 2, the bleeding amount detection part 315 detects a bleeding amount (or bleeding speed) from the partial moving image (S421), and the playback part 314 can reduce the playback speed as the bleeding amount (or bleeding speed) is large (or high) (S422). At step S407, the partial moving image is played back at an adjusted playback speed. Thus, the scene at a time when bleeding occurs (at an event occurrence time) can be played back slowly when a large amount of bleeding or bleeding at high speed occurs, resulting in easy and reliable grasping of the bleeding site and the cause of the bleeding.

Note that the playback part 314 may change the playback speed only in the case where the playback mode is in the slow playback, or may change the playback speed for all the playback modes.

### Embodiment 3

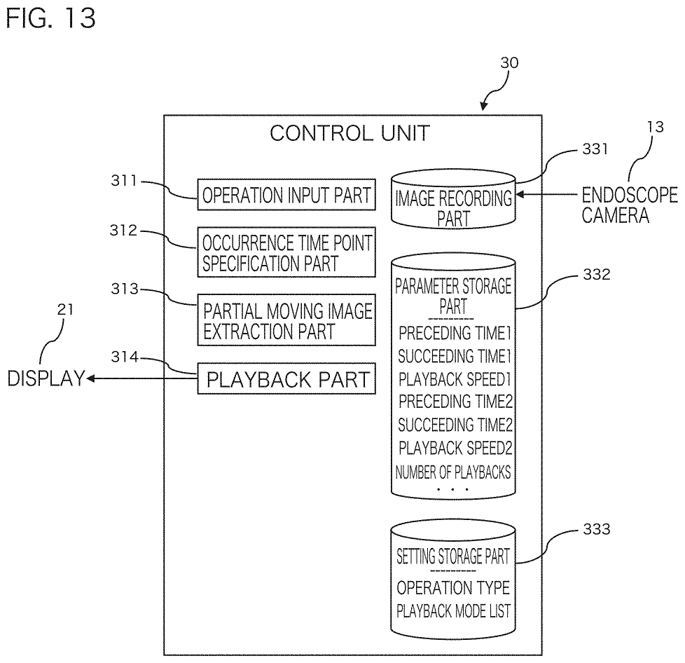

FIG. 13 is an explanatory view illustrating an example of the software configuration of a control unit 30 according to Embodiment 3. Though the playback speed of a partial moving image is assumed to be constant in Embodiment 1 described above, the playback speed in a short period of time before and after the event occurrence time of the partial moving image can be reduced in Embodiment 3.

The control unit 30 according to Embodiment 2 stores two sets of preceding times, succeeding times and playback speeds as parameters to be stored in the parameter storage part 332 unlike the configuration of the control unit 30 according to Embodiment 1 described above (see FIG. 4). In the example of FIG. 13, a first set of a preceding time 1 (i1(1)), a succeeding time 1 (i2(1)) and a playback speed 1 and a second set of a preceding time 2 (i1(2)), a succeeding time (i2(2)) and a playback speed 2 are registered in the parameter storage part 332. A period for the second set is shorter than a period for the first set, and both of the periods for the first set and the second set contain an event start time (frame F1). Furthermore, the playback speed 2 for the second set is lower than the playback speed 1 for the first set.

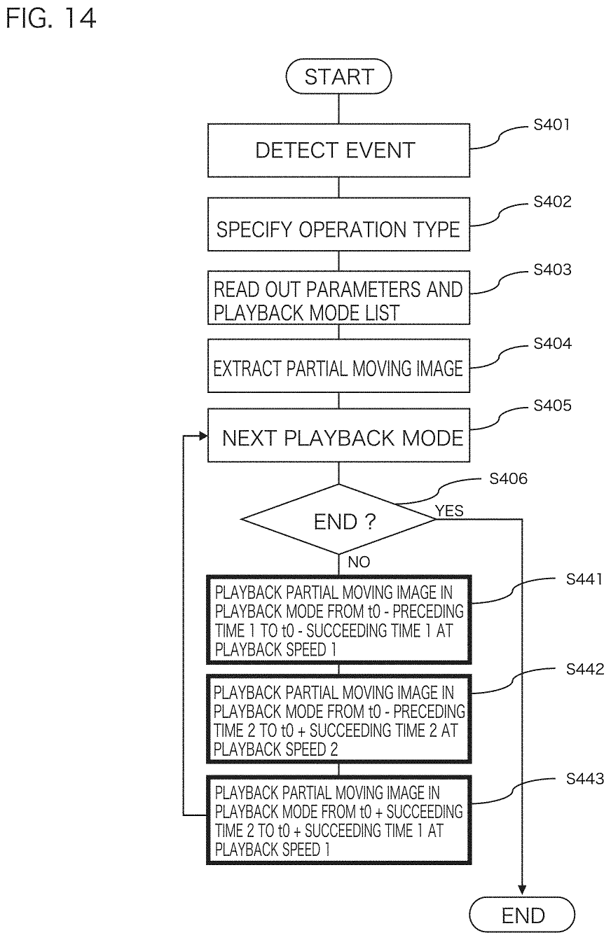

FIG. 14 is a flowchart of the processing executed by the control unit 30 according to Embodiment 3. The difference from Embodiment 1 (FIG. 10) will be described. In Embodiment 3, step S441 or S443 is executed in place of the playback processing at step S407. At step S441, a playback is made at the playback speed 1 for the first set for the duration of a period from the start time t0−i1 (1)ₐₛ in the start time t0−i1 according to Embodiment 1 to the start time of the second set t0−i1(2) (S441), a playback is made at the playback speed 2 for the second set for the duration of a period from the start time of the second set t0−i1(2) to the end time t0+i2 (2) of the second set (S442), and a playback is made at the playback speed 1 for the first set for the duration of a period from the end time t0+i2 (2) of the second set to the end time of the first set t0+i2(1) (S443). Thus, the slow playback is made possible during the period for the second set that is shorter than the first set and includes the event occurrence time.

In Embodiment 3 as well, the bleeding amount detection part 315 is provided as in Embodiment 2, and a playback may be made at the playback speed 2 depending on the bleeding amount or the bleeding speed during the period for the second set.

Furthermore, the playback speed 1 for the first set is set to a speed higher than the normal speed (real time) to perform a fast-forward playback the scenes other than the scene including the event occurrence time.

### Embodiment 4

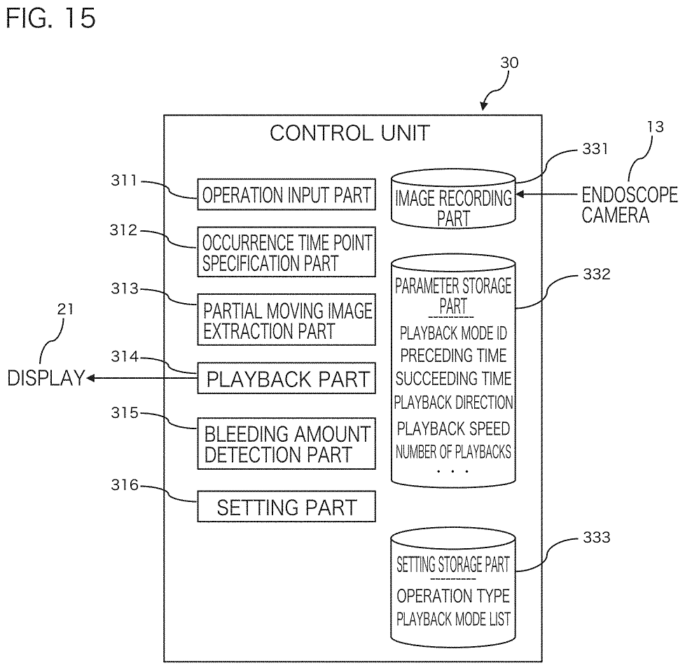

FIG. 15 is an explanatory view illustrating an example of the software configuration of a control unit 30 according to Embodiment 4. In Embodiment 4, settings of various parameters (preceding time, succeeding time, playback direction, playback speed, the number of playbacks and the like) related to the playback mode are made changeable. In Embodiment 4, the parameter storage part 332′ stores various parameters in association with the playback mode IDs indicating the playback modes. Furthermore, the control unit 30 has a setting part 316 that accepts input of the parameters to be stored in the parameter storage part 332′. The setting part 316 allows for previous registration of the parameters and changes in the parameters during a surgical operation or during a playback of the moving image as well.

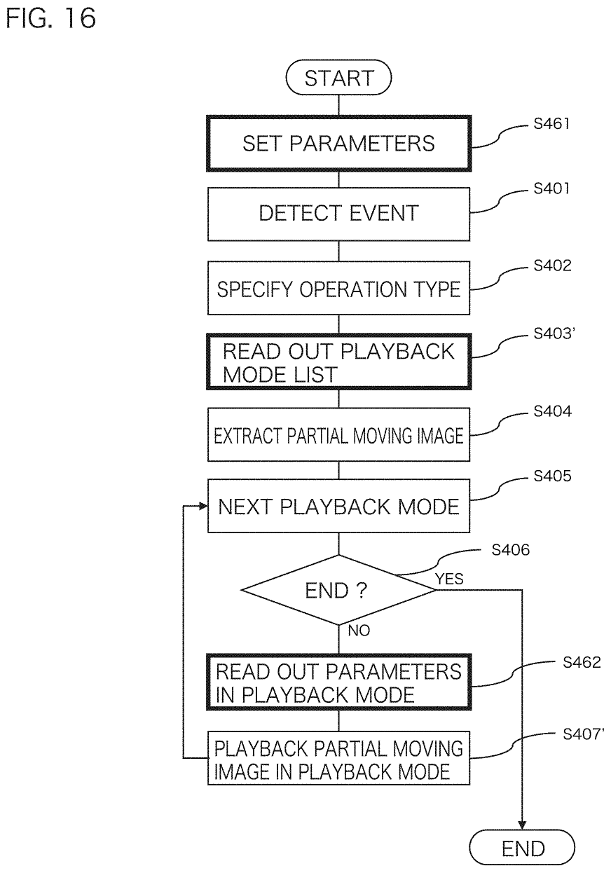

FIG. 16 is flowchart of the processing executed by the control unit 30 according to Embodiment 4. The difference from the processing according to Embodiment 1 (FIG. 10) will be described. In Embodiment 4, the setting part 316 accepts input of the parameters for each playback mode and registers them in the parameter storage part 332′ (S461). At step S403, only the playback mode list is read (S403′), and before playback step S407, the playback part 314 reads out parameters corresponding to the playback mode from the parameter storage part 332′ (S462) and plays back a partial moving image using the read parameters (S407′).

Thus, a playback speed and a playback period depending on the playback modes can freely be set.

### Embodiment 5

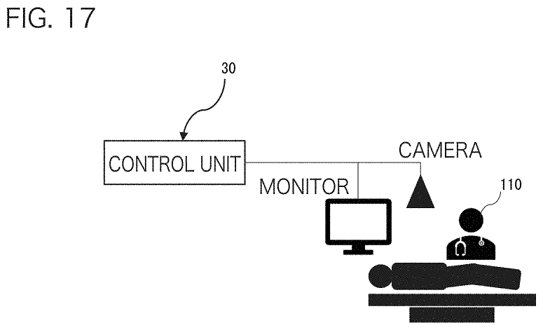

FIG. 17 is an explanatory view illustrating an overview of a surgical operation system 1 according to Embodiment 5. In Embodiment 5, it is assumed that the operating surgeon 110 directly operates a surgical instrument to perform procedures on the patient 100 instead of remote control. Examples of the surgical operation may be any of an abdominal operation and a laparoscopic surgery. Even in this case as well, the control unit 30 according to the above-described embodiments can play back the scene including the event occurrence time multiple times while varying the playback modes.

### Embodiment 6

In Embodiment 6, a configuration in which a partial moving image taken during a predetermined time range including the bleeding start time is played back in the case where a predetermined or larger amount of bleeding is detected. Note that, since the software configuration of the control unit 30 is similar to that of Embodiment 2, the description thereof is not repeated here.

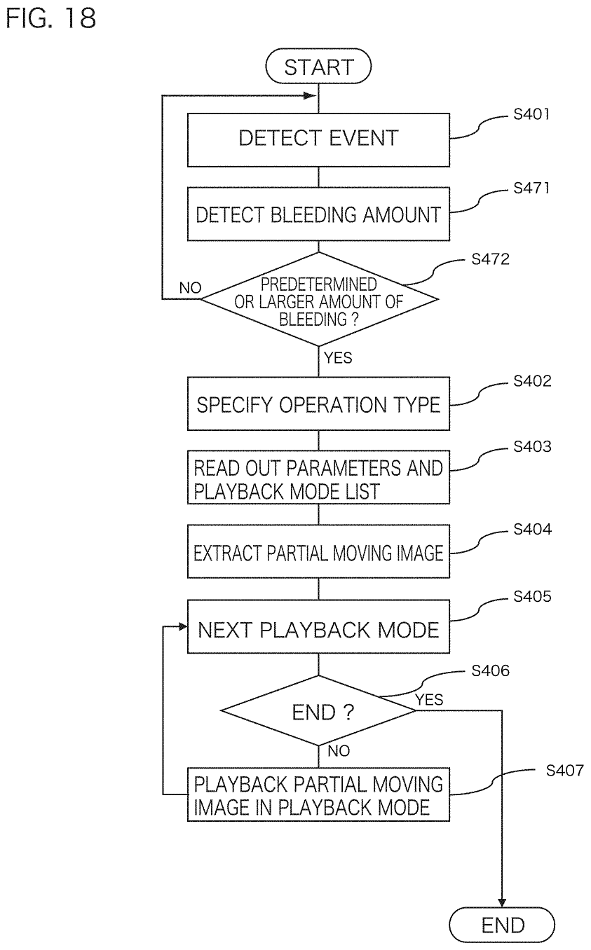

FIG. 18 is a flowchart of the processing executed by a control unit 30 according to Embodiment 6. The difference from the processing according to Embodiment 1 (FIG. 10) will be described. In the case where an event (bleeding in the present embodiment) is detected at step S401, the bleeding amount detection part 315 analyzes the images of the frames F obtained by the endoscope camera 13 and detects the amount of bleeding (S471). The bleeding amount detection part 315 may detect the amount of bleeding depending on the number of pixels having a color of blood and on the area dimension of such a region, as described in Embodiment 2. The bleeding amount detection part 315 detects the amount of bleeding every time an image of each of the frames F is input, and determines the presence or absence of a predetermined or larger amount of bleeding by comparing the detected bleeding amount with a preset threshold (S472). When a predetermined or larger amount of bleeding is absent (S472: NO), the control unit 30 returns the processing to step S401.

When determining that a predetermined or larger amount of bleeding is present (S472: YES), the control unit 30 executes the processing at and after step S402. That is, the control unit 30 reads out various parameters (playback speed, the number of repetitions for the repetitive playback and the like) related to playback modes and a playback mode list according to the operation type such as resection, abrasion, traction, suture, suction and the like and plays back a partial moving image extracted from the recorded image M1 based on the read parameters and the playback mode list. The extraction method of the partial moving image is similar to that of Embodiment 1. That is, the partial moving image extraction part 313 is only required to extract frames F included in the time range from the time before start of bleeding (t0−i1) to the time after bleeding (t0+i2) as a partial moving image in the recorded image M1 stored in the image recording part 331. In the present embodiment, t0 represents the bleeding start time. The times i1, i2 may previously be set, or may be set according to the operation type such as resection, abrasion, traction, suture and suction and the like as well as the shot scene or the like.

The playback part 314 plays back the partial moving image according to the playback modes including at least one of the normal playback, the slow playback, the frame-by-frame playback, the repetitive playback and the reverse playback. The playback part 314 may output a partial image as a wipe image W as illustrated in FIG. 9 superimposed on the real-time image (operation field images) acquired by the endoscope camera 13, or may output the partial image to a sub display having been provided separately from the display 21. Furthermore, the playback part 314 may repetitively play back the partial moving image while switching between at least two of the normal playback, the slow playback, the frame-by-frame playback and the reverse playback. Moreover, the playback part 314 may play back the scene including the bleeding start time at a playback speed lower than that of the scene before and after the bleeding starts.

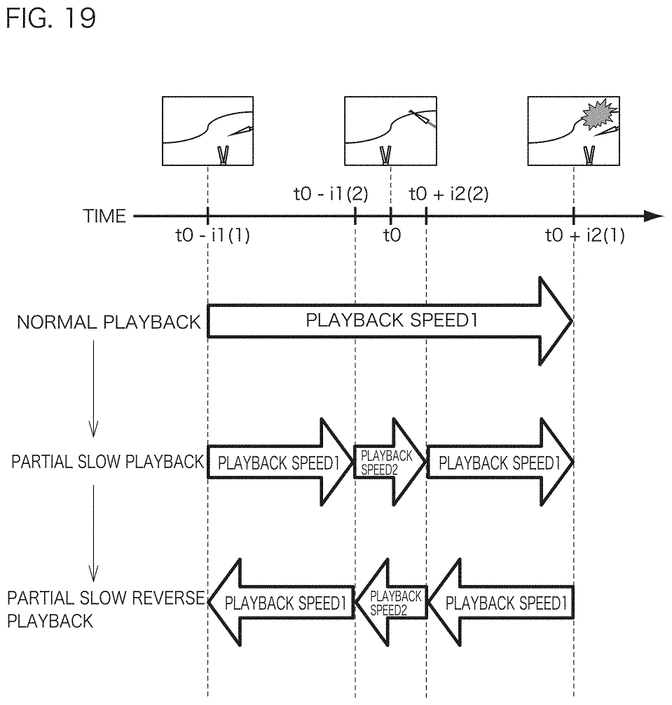

FIG. 19 is an explanatory view illustrating one example of a playback mode. In the case where the time t=t0 is assumed as the bleeding start time for the recorded image M1 including the scene where a predetermined or larger amount of bleeding is detected, the partial moving image extraction part 313 extracts the frames F in the time range from the time t=t0−i1(1) to t=t0+i2(1) as a partial moving image. For example, the time i1(1) is set as 2 to 6 seconds, for example, depending on the operation type and the scene. For example, the time i2(1) is set as an appropriate time such as 2 seconds.

The playback part 314 plays back the partial moving image by switching the playback modes from the normal playback, the partial slow playback and the partial slow reverse playback in this order. The playback part 314 may repeat this order from the normal playback, the partial slow playback and the partial slow reverse playback after the partial slow reverse playback.

In the normal playback, the time i1 (1) is set as 6 seconds while the time i2 (1) is set as 2 seconds, for example. That is, the playback part 314 plays back a partial moving image of 8 seconds in total from 6 seconds before the bleeding start time t0 2 seconds after the bleeding start time at the normal playback speed (playback speed 1). The surgical image shot before bleeding is played back a little longer, which allows the operator to grasp the operation that causes the bleeding.

In the partial slow playback, the time i1(1) is set as 3 seconds, the time i2(1) is set as 2 seconds and the times i1(2) and i2(2) are set as 0.2, for example. That is, the playback part 314 plays back a partial moving image of 2.8 seconds from 3 to 0.2 seconds before the bleeding start time at the normal playback speed (playback speed 1), and plays back in slow motion a partial moving image of 0.4 seconds, i.e. from 0.2 seconds before the bleeding start time t0 0.2 seconds after the bleeding start time, at one fifths of the normal playback speed (playback speed 2). Note that the playback speed in the slow playback may be set to an appropriate speed, not limited to one fifth of the normal speed. After the slow playback, the playback part 314 plays back a partial moving image of 1.8, i.e. seconds from 0.2 to 2 seconds after the bleeding start time, at the normal playback speed (playback speed 1). In the partial slow playback, the instant of bleeding is played back in slow motion, and thus the operator has an advantage of easily recognizing a bleeding site. Furthermore, the playback mode is changed immediately before bleeding, which allows the operator to predict that bleeding is going to occur. Moreover, a partial slow playback is employed, which avoids extension of the playback time.

The time setting in the partial slow reverse playback is similar to that in the partial slow playback. The playback part 314 reversely plays back a partial moving image from 0.2 to 2 seconds after the bleeding start time at the normal playback speed (playback speed 1), and reversely plays back in slow motion a partial moving image from 0.2 seconds before the bleeding start time t0 0.2 seconds after the bleeding start time at one fifths of the normal playback speed (playback speed 2). Note that the playback speed in the slow reverse playback may be set to an appropriate speed, not limited to one fifth of the normal speed. After the slow playback, the playback part 314 reversely plays back a partial moving image from 3 to 0.2 seconds before the bleeding start time at the normal playback speed (playback speed 1). Since the partial slow reverse playback enables image display such that spread blood gradually converges to one location, the operator can easily recognize a bleeding site by grasping the convergent point.

In FIG. 19, the configuration of playing back a partial image in the normal playback, the partial slow playback and the partial slow reverse playback in this order is described. Such a display order and display modes may be set to the control unit 30 as a default. Furthermore, the control unit 30 may accept changes in the playback order and the playback modes through the operation unit 20. The playback order and playback modes may be set by an operator as appropriate. For example, the operator may change settings such that a partial moving image is played back according to the partial slow playback and the partial slow reverse playback in this order or may change settings such that a partial moving image is played back according to the normal playback, the partial slow playback, the reverse playback and the partial slow reverse playback in this order. In the case of accepting changes in the playback order and the playback modes, the control unit 30 plays back a partial image according to the changed playback order and playback modes.

As described above, since a bleeding scene is displayed in image only in the case where a predetermined or larger amount of bleeding is detected in Embodiment 6, only the bleeding scene that is to be recognized by the operator can be displayed without hindering the surgical operation.

Though the threshold for the bleeding amount is assumed to be previously set in Embodiment 6, setting of the threshold by the operator such as an operating surgeon may be accepted through the operation input part 311. When accepting the setting of the threshold, by comparing the amount of bleeding estimated from the image and the set threshold, the bleeding amount detection part 315 may determine the presence or absence of a predetermined or larger amount of bleeding.

### Embodiment 7

In Embodiment 7, bleeding equal to or more than a threshold that occurs during image capturing is recognized, and the recognized bleeding scene is displayed as a thumbnail on the display 21. When accepting a selection of the thumbnail, the control unit 30 plays back a moving image of the corresponding bleeding scene.

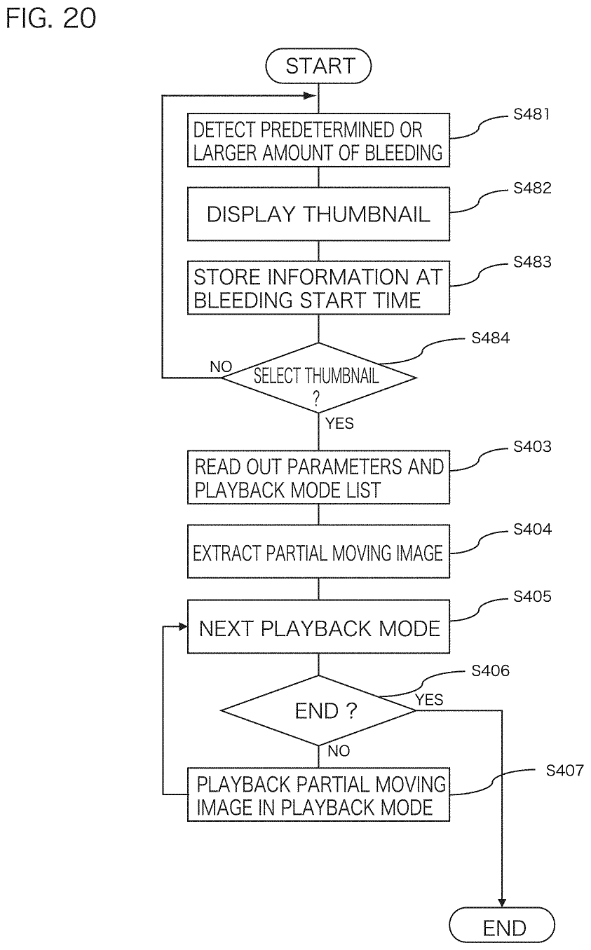

FIG. 20 is a flowchart of the processing executed by the control unit 30 according to Embodiment 7. The difference from the processing in Embodiment 6 (FIG. 18) will be described. When detecting a predetermined or larger amount of bleeding is detected according to the same procedure as that of Embodiment 6 (S481), the control unit 30 generates a thumbnail showing the bleeding scene and displays the generated thumbnail on the display 21 such that it is superimposed on the operation field image (S482). As a thumbnail, a still picture image or a moving image representative of the bleeding scene may be displayed. Furthermore, the control unit 30 stores in the parameter storage part 332 the information at bleeding start time t0, for example, as information for specifying bleeding scene in association with the thumbnail (S483).

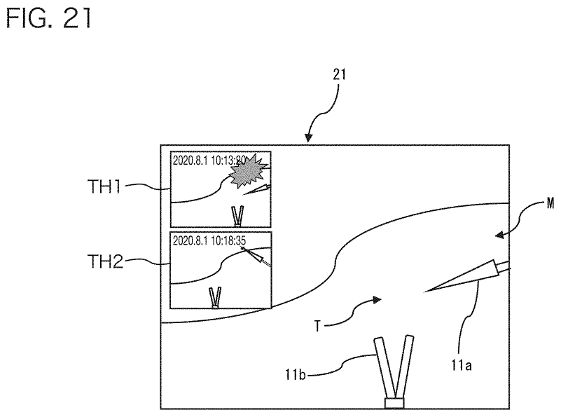

FIG. 21 is an explanatory view illustrating a display example of a thumbnail showing a bleeding scene. FIG. 21 illustrates an example in which two thumbnails TH1 and TH2 are displayed on the display 21 so as to be superimposed on the operation field image. The thumbnails TH1, TH2 are preferably arranged so as to avoid the area close to the center of the operation field images in order that the recognition of a treated region is not hindered. The control unit 30 thus has to display the generated thumbnails TH1, TH2 such that they are arranged at the peripheral region of the operation field images, for example. Alternatively, the control unit 30 may grasp the position of the surgical instruments 11a, 11b by image analysis and display the thumbnails TH1, TH2 so as to avoid the grasped positions of the surgical instruments 11a, 11b. Furthermore, the control unit 30 may add information on the detection time when a predetermined or larger amount of bleeding is detected to the thumbnails TH1, TH2.

The control unit 30 determines whether or not a selection of a thumbnail is accepted through the operation input part 311 (S484). The operation input part 311 may accept a selection performed on the thumbnail by using the input device such as a joystick, a foot pedal and the like included in the controller part 22, or may accept voice instructions for selecting a thumbnail using a microphone. When not accepting a selection (S484: NO), the control unit 30 returns the processing to step S481.

The control unit 30 adds a new thumbnail to the operation field images every time a predetermined or larger amount of bleeding is detected. Note that since the excessive number of the thumbnails makes it difficult to check the affected area T in the operation field images, the number of thumbnails to be displayed may be limited. For example, the control unit 30 may select a predetermined number of thumbnails from the thumbnail having the latest detection time in order. Moreover, instead of a configuration in which a thumbnail is displayed so as to be superimposed on the operation field images, the operation field images may be displayed on the display 21 while the thumbnail may be configured to be separately displayed on a sub display.

When accepting a selection operation performed on the thumbnail (S484: YES), the partial moving image extraction part 313 respectively reads out the parameters and the playback mode list from the parameter storage part 332 and the setting storage part 333 for the bleeding scene shown by the selected thumbnail (S403).

Here, the partial moving image extraction part 313 can read the necessary parameters and playback mode list using the information of the bleeding start time t0 associated with the thumbnail as a retrieval key.

The processing after reading the parameters and the playback mode list is similar to that of Embodiment 6. The partial moving image extraction part 313 extracts a partial moving image, and the playback part 314 plays back the partial moving image according to the playback mode described in the playback mode list. The playback part 314 may be only required to playback the partial moving image according to the playback mode including at least one of the normal playback, the slow playback, the frame-by-frame playback, the repetitive playback and the reverse playback. The playback part 314 may output the partial image as a wipe image W as illustrated in FIG. 9 superimposed on the real-time image (operation field images) acquired by the endoscope camera 13, or may output the partial image to a sub display having been provided separately from the display 21. Furthermore, the playback part 314 may repetitively playback the partial moving image by switching between at least two of the normal playback, the slow playback, the frame-by-frame playback and the reverse playback. Moreover, the playback part 314 may play back the scene including the bleeding start time at a speed lower than that of the scene before and after the bleeding starts.

Since, in the description above, a moving image of a bleeding scene is played back only in the case where a thumbnail is selected in the surgical operation system 1 according to Embodiment 7, only the bleeding scene that is desired by an operator such as an operating surgeon can be offered as a moving image.

### Embodiment 8

In Embodiment 8, a configuration in which detection of bleeding is notified in the case where bleeding is detected at a peripheral region of the operation field region acquired by the endoscope camera 13 is described.

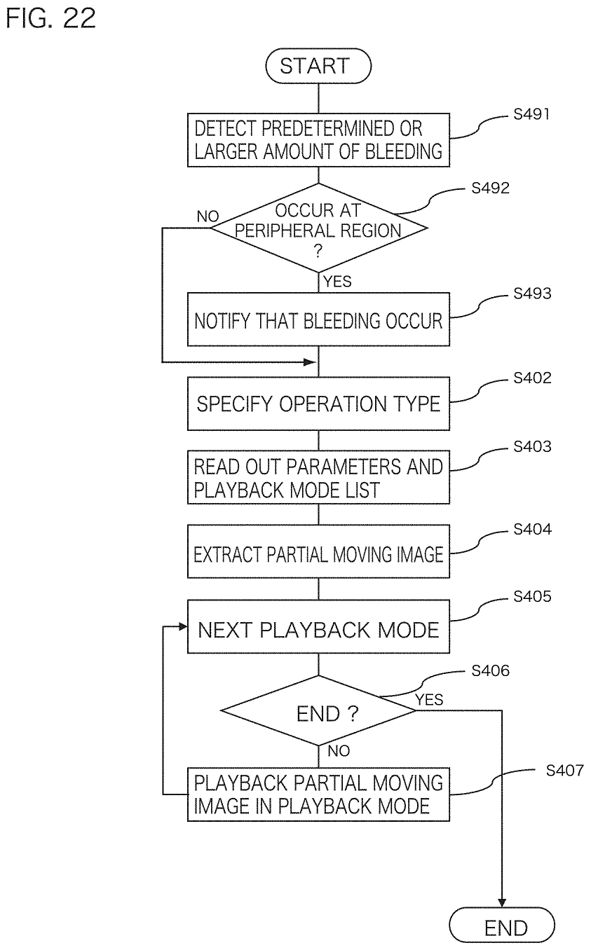

FIG. 22 is a flowchart of the processing executed by a control unit 30 according to Embodiment 8. The difference from the processing in Embodiment 6 (FIG. 18) will be described. When detecting a predetermined or larger amount of bleeding is detected according to the same procedure as Embodiment 6 (S491), the control unit 30 determines whether or not such bleeding occurs at the peripheral region of the operation field images (S492). The peripheral region may be set to a region except for the vicinity of the center (target region) of the operation field images. In one example, the peripheral region corresponds to the upper, lower, left or right quarter of the operation field images.

When determining that bleeding occurs at the peripheral region of the operation field images (S492: YES), the control unit 30 notifies of information that bleeding occurs (S493). The control unit 30 displays text information indicating the occurrence of bleeding, an icon or the like, for example, on the display 21. Alternatively, the control unit 30 may output voice information indicating the occurrence of bleeding through the speaker 23.

Since, in the description above, information that bleeding occurs is notified in the case where bleeding occurs at the peripheral region of the operation field images in Embodiment 8, the operator can recognize the presence of bleeding that is hard to notice.

### Embodiment 9

In Embodiment 9, a configuration in which a graph showing a chronological variation of the amount of bleeding is displayed is described.

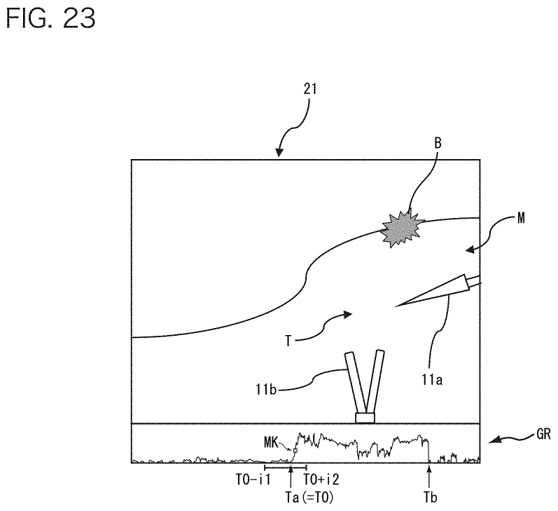

FIG. 23 is a display example of the graph showing the chronological variation of the amount of bleeding. The bleeding amount detection part 315 of the control unit 30 can detect the amount of bleeding at each of the times by analyzing the frame F at each of the times. The control unit 30 may generate the graph showing the chronological variation of the amount of bleeding and display it together with the operation field images. FIG. 23 illustrates an example in which a graph GR showing the chronological variation of the amount of bleeding is displayed below the operation field images. The horizontal axis of the graph GR represents the time while the vertical axis thereof represents the amount of bleeding. In this graph GR, the time Ta represents a bleeding start time while the time Tb represents a bleeding stop time. In place of the amount of bleeding, the chronological variation of the number of pixels or the area equivalent to bleeding may be displayed in a graphical form.

Furthermore, the control unit 30 may denote a point on the graph GR where the amount of bleeding is equal to or more than a predetermined amount by a mark MK or the like as illustrated in FIG. 23. Moreover, the control unit 30 may highlight the part on the graph where the amount of bleeding is equal to or more than a predetermined amount by a bold line or change in color for clear expression thereof. Additionally, the control unit 30 may display a playback section (t0·i1≤t≤t0+i2) of the partial moving image together with the graph GR. In addition, the control unit 30 may display the graph GR during playback of the partial moving image.

Since, in the description above, the graph representing the chronological variation of the amount of bleeding is displayed in Embodiment 9, the operator can grasp a bleeding state that occurred in the past.

### Embodiment 10

In Embodiment 10, a configuration is described in which determination as to whether or not a predetermined or larger amount of bleeding occurs is performed in the case where a resection instrument is included in the operation field images while such a determination is not performed in the case where a hemostasis device is included in the operation field images.

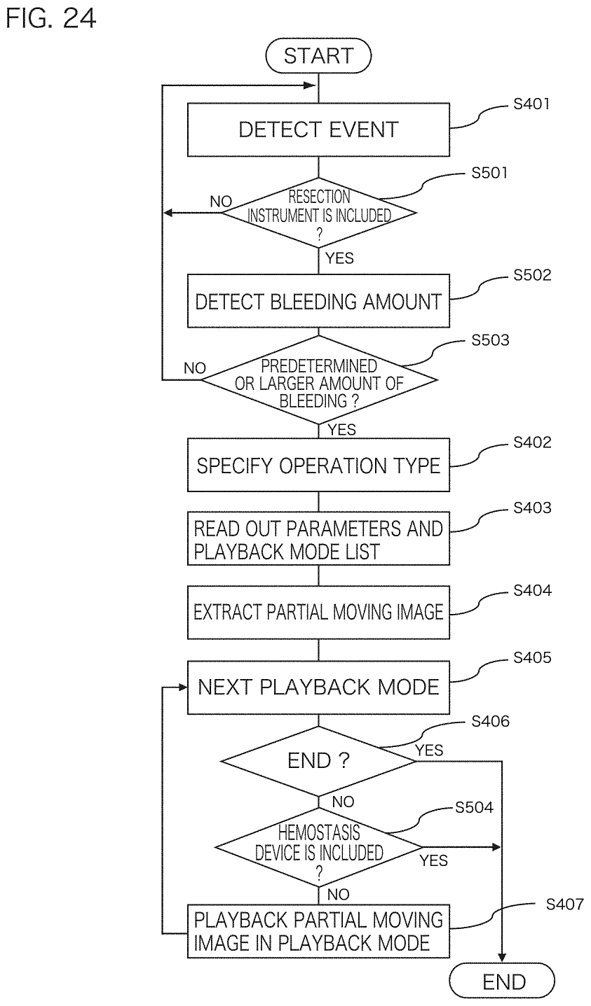

FIG. 24 is a flowchart of the processing executed by a control unit 30 according to Embodiment 10. The difference from the processing in Embodiment 1 (FIG. 10) will be described. When detecting an event (bleeding in the present embodiment) at step S401, the control unit 30 determines whether or not a resection instrument is included in the operation field images by analyzing the images of the frames F acquired by the endoscope camera 13 (S501). The control unit 30 can determine whether or not a resection instrument is included in the operation field images by judging whether or not a figure of a preset shape is included by using a method of a template matching or the like. When the resection instrument is not included (S501: NO), the control unit 30 returns the processing to step S401.

When the resection instrument is included (S501: YES), the control unit 30 detects the amount of bleeding by analyzing the images of the frames F (S502). The bleeding amount detection part 315 may detect the amount of bleeding depending on the number of pixels having a color corresponding to blood and on the area dimension of the bleeding region, as described in Embodiment 2. The bleeding amount detection part 315 detects the amount of bleeding every time the image of the frame F is input, and determines the presence or absence of a predetermined or larger amount of bleeding by comparing the detected bleeding amount and a preset threshold (S503). When a predetermined or larger amount of bleeding is absent (S503: NO), the control unit 30 returns the processing to step S401.

When a predetermined or larger amount of bleeding is present (S503: YES), the control unit 30 executes the processing at and after step S402 described in Embodiment 1. That is, the control unit 30 reads out various parameters related to playback modes (playback speed, the number of repetitions for the repetitive playback and the like) according to the operation type such as resection, abrasion, traction, suture, suction and the like as well as a playback mode list and plays back a partial moving image extracted from the recorded image M1 based on the read parameters and the playback mode list. The extraction method of the partial moving image is similar to that of Embodiment 1. In other words, the partial moving image extraction part 313 is only required to extract the frames F included in the time range from the time before bleeding (t0·i1) to the time after the bleeding (t0+i2) as a partial moving image among the recorded image Ml. In the present embodiment, the time t0 represents the bleeding start time. The time i1, i2 may previously be set or may be set according to the operation type such as resection, abrasion, traction, suture, suction and the like as well as the shot scene.

Furthermore, when playing back a partial moving image, the control unit 30 analyzes the images of the frames F acquired by the endoscope camera 13 and determines whether or not the hemostasis device is included in the operation field images (S504). The control unit 30 can determine whether or not the hemostasis device is included in the operation field images by judging whether or not a figure of a preset shape is included by using a method of a template matching or the like. When the hemostasis device is not included (S504: NO), the control unit 30 plays back the partial moving image (S407). Meanwhile, when the hemostasis device is included (S504: YES), the control unit 30 ends the processing according to the flowchart without performing playback of the partial moving image.

As described above, in Embodiment 10, the partial moving image is played back only in the case where the amount of bleeding becomes equal to or larger than a predetermined amount during resection of the region to be operated using the resection instrument. Moreover, when the hemostasis device appears in the operation field images, a hemostasis site appears to be determined and playing back the partial moving image is controlled so as not to be performed.

While the present embodiments have been described above, such embodiments are for easy understanding of the present invention, not for limitative understanding of the present invention. It is understood that changes and variations may be made without departing from the spirit of the invention and the equivalence thereof is also embraced.

Though a case where the event is regarded as bleeding B from a region near the affected area T of the patient 100 is described in the above-described embodiments, the event may be regarded as detection of damage, malpractice and other abnormalities, for example.

The present surgical operation system can be applied to all the procedures having a possibility of causing the above-described accidents as well as a surgical operation. The examples include any procedures including accidents caused by or suspected to be caused by medical treatment such as a physical examination, a test, a diagnostic paracentesis, specimen collection, imaging study, administration/injection (including a blood transfusion), rehabilitation, anesthesia, radiation therapy and use of a medical equipment.

The number of the surgical operation systems in a surgical operation may be more the one, not limited to one. Here, the motion of the operation unit and the work unit are synchronized with each other, or may be configured to separately function (a first operation device and a second operation device do not coordinate with each other).

Furthermore, a subject to be operated in the present system may be animals, objects as a subject for training, plants or the like, not limited to the human body.

Moreover, in each of the above-mentioned embodiments, detection of digestive fluid or bile drained due to damage to the internal organs or the digestive organs in place of detection of the amount of bleeding can produce a similar working effect.

Additionally, though the event is assumed as an incident such as bleeding in each of the above-mentioned embodiments, a specific operation with a surgical instrument, for example, can be detected as an event. For example, suturing or anastomosing using a suturing instrument (stapler) is detected as an event, and the situation during suturing or anastomosing can be played back. This makes it possible to confirm whether or not the treatment is properly performed.

Though the present embodiment is assumed to be applied to a real-time moving image shot during a surgical operation performed on the patient 100, it is also useful in the situation where the recorded image M1 recording the situation of the surgical operation is viewed later. For example, the operating surgeon 110 may view the recorded image M1 of the surgical operation performed by himself/herself or another surgeon for using the cause or location of an event occurrence as reference for future surgical operations.

It is noted that, as used herein and in the appended claims, the singular forms “a”, “an”, and “the” include plural referents unless the context clearly dictates otherwise.

1-9. (canceled)

10. A non-transitory recording medium recoding a program that causes a computer to execute processing comprising:
recording operation field images obtained by chronologically shooting an operation field under endoscopic surgery;
determining presence or absence of a predetermined or larger amount of bleeding based on the operation field images; and
playing back partial images according to a set playback mode in a time range from a time before a start of bleeding to the time after the bleeding among the recorded operation field images, when it is determined that the predetermined or larger amount of bleeding is present.

11. The non-transitory recording medium according to claim 10, wherein the playback mode includes at least one of a normal playback, a slow playback, a frame-by-frame playback, a repetitive playback and a reverse playback.

12. The non-transitory recording medium according to claim 10, wherein the playback mode includes a repetitive playback while switching between at least two of a normal playback, a slow playback, a frame-by-frame playback and a reverse playback.

13. The non-transitory recording medium according to claim 10, wherein the playback mode includes a mode in which a playback speed of a scene including a start time of bleeding is made lower than the playback speed of the scene before and after the start of bleeding.

14. The non-transitory recording medium according to claim 10, causing the computer to further execute processing of:
displaying the operation field images shot during the endoscopic surgery on one display area and playing back the partial images of the time range on another display area.

15. The non-transitory recording medium according to claim 10, causing the computer to further execute processing of:
displaying, in a case where a plurality of scenes of a predetermined or larger amount of bleeding are detected, thumbnails representing respective bleeding scenes, and
playing back, in a case where a selection for the displayed thumbnails is accepted, the partial images of a corresponding bleeding scene according to the playback mode.

16. The non-transitory recording medium according to claim 10, causing the computer to further execute processing of:
notifying of, in a case where bleeding is detected at a peripheral region of the operation field images, a fact that bleeding has been detected.

17. The non-transitory recording medium according to claim 10, causing the computer to further execute processing of:
displaying a graph representing a chronological variation of a bleeding amount.

18. An image playback method using a computer comprising:
recording operation field images obtained by chronologically shooting an operation field under endoscopic surgery;
determining presence or absence of a predetermined or larger amount of bleeding based on the operation field images; and
playing back partial images according to a set playback mode in a time range from a time before a start of bleeding to the time after the bleeding among the recorded operation field images, when it is determined that the predetermined or larger amount of bleeding is present.
